(19) United States
(12) Patent Application Publication
(10) Pub. No.: US 2010/0233771 A1
(43) Pub. Date: Sep. 16, 2010

(54) SYSTEM FOR PRE-TREATMENT OF BIOMASS FOR THE PRODUCTION OF ETHANOL

(76) Inventors: William F. McDonald, Utica, OH (US); Jason Richard Kwiatkowski, Alton, IL (US); Neelakantam V. Narendranath, Sioux Falls, SD (US); Jason Alan Bootsma, Sioux Falls, SD (US); David Charles Carlson, Yankton, SD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(21) Appl. No.: 12/716,984

(22) Filed: Mar. 3, 2010

Related U.S. Application Data
(60) Provisional application No. 61/157,146, filed on Mar. 3, 2009.

(51) Int. Cl. C12P 7/06; C12M 1/00

(52) U.S. Cl. 435/161; 435/289.1

(57) ABSTRACT

A system for the pre-treatment of biomass for use in a biorefinery to produce ethanol and other bioproducts is disclosed. The system comprises a method comprising the steps of preparing the biomass into prepared biomass; pre-treating the prepared biomass into pre-treated biomass by application of a dilute acid having a concentration of about 0.8 to 1.1 percent by weight at a temperature of about 130 to about 170 degrees Celsius for a period of time in a range of about 8 to 12 minutes. The system also comprises an apparatus for separating the pre-treated biomass into a liquid component comprising pentose and a solids component comprising cellulose and lignin; a fermentation system configured to produce the fermentation product; and a distillation system. The fermentation product comprises ethanol. The biomass comprises lignocellulosic material, comprising corn cobs, corn plant husks, corn plant leaves and corn plant stalks.

### CROSS-REFERENCE TO RELATED APPLICATIONS

The present application claims priority to and incorporates by reference the following application: U.S. Provisional Application Ser. No. 61/157,146, titled Dilute Acid Pretreatment of Biomass for the Production of Ethanol, filed on Mar. 3, 2009.

The present application relates to and incorporates by reference the following applications: (a) U.S. Application Ser. No. (Atty. Docket No. P184 1260.1), titled System for Treatment of Biomass to Facilitate the Production of Ethanol, filed on Mar. 3, 2010; and (b) U.S. Application Ser. No. (Atty. Docket No. P184 1280.1), titled System for Fermentation of Biomass for the Production of Ethanol, filed on Mar. 3, 2010.

### FIELD

The present invention relates to a system for pre-treatment of biomass for the production of ethanol. The present invention also relates to a system for pre-treatment of biomass in the form of corn cobs and/or stover for the production of cellulosic ethanol and recovery of other bioproducts. The present invention further relates to a system for the pre-treatment of biomass in the form of corn cobs and/or stover with acid prior to fermentation of the biomass into cellulosic ethanol.

### BACKGROUND

Ethanol can be produced from grain-based feedstocks (e.g. corn, sorghum/milo, barley, wheat, soybeans, etc.), from sugar (e.g. from sugar cane, sugar beets, etc.), and from biomass (e.g. from lignocellulosic feedstocks such as switchgrass, corn cobs and stover, wood or other plant material).

Biomass comprises plant matter that can be suitable for direct use as a fuel/energy source or as a feedstock for processing into another bioproduct (e.g., a biofuel such as cellulosic ethanol) produced at a biorefinery (such as an ethanol plant). Biomass may comprise, for example, corn cobs and stover (e.g., stalks and leaves) made available during or after harvesting of the corn kernels, fiber from the corn kernel, switchgrass, farm or agricultural residue, wood chips or other wood waste, and other plant matter (grown for processing into bioproducts or for other purposes). In order to be used or processed, biomass will be harvested and collected from the field and transported to the location where it is to be used or processed.

In a conventional ethanol plant producing ethanol from corn, ethanol is produced from starch. Corn kernels are cleaned and milled to prepare starch-containing material for processing. (Corn kernels can also be fractionated to separate the starch-containing material (e.g. endosperm) from other matter (such as fiber and germ).) The starch-containing material is slurried with water and liquefied to facilitate saccharification where the starch is converted into sugar (e.g. glucose) and fermentation where the sugar is converted by an ethanologen (e.g. yeast) into ethanol. The product of fermentation (i.e. fermentation product) is beer, which comprises a liquid component containing ethanol and water and soluble components, and a solids component containing unfermented particulate matter (among other things). The fermentation product is sent to a distillation system. In the distillation system, the fermentation product is distilled and dehydrated into ethanol. The residual matter (e.g. whole stillage) comprises water, soluble components, oil and unfermented solids (i.e. the solids component of the beer with substantially all ethanol removed that can be dried into dried distillers grains (DDG) and sold as an animal feed product). Other co-products, for example syrup (and oil contained in the syrup), can also be recovered from the stillage. Water removed from the fermentation product in distillation can be treated for re-use at the plant.

In a biorefinery configured to produce ethanol from biomass, ethanol is produced from lignocellulosic material. Lignocellulosic biomass typically comprises cellulose, hemicellulose and lignin. Cellulose (a type of glucan) is a polysaccharide comprising hexose (C6) sugar monomers such as glucose linked in linear chains. Hemicellulose is a branched chain polysaccharide that may comprise several different pentose (C5) sugar monomers (such as xylose and arabinose) and small amounts of hexose (C6) sugar monomers in branched chains.

The biomass is prepared so that sugars in the lignocellulosic material (such as glucose from the cellulose and xylose from the hemicellulose) can be made accessible and fermented into a fermentation product from which ethanol can be recovered. After fermentation the fermentation product is sent to the distillation system, where the ethanol is recovered by distillation and dehydration. Other bioproducts such as lignin and organic acids may also be recovered as byproducts or co-products during the processing of biomass into ethanol. Determination of how to more efficiently prepare and treat the biomass for production into ethanol will depend upon the source and type or composition of the biomass. Biomass of different types or from different sources is likely to vary in properties and composition (e.g. relative amounts of cellulose, hemicellulose, lignin and other components). For example the composition of wood chips will differ from the composition of corn cobs or switchgrass.

It would be advantageous to provide for a system for pre-treatment of biomass such as corn cobs and/or stover for the production of cellulosic ethanol. It would also be advantageous to provide for a system for the pre-treatment of biomass in the form of corn cobs and/or stover with acid in order to facilitate improvement in the efficiency and yield of cellulosic ethanol from the biomass.

### SUMMARY

The present invention relates to a method to pre-treat biomass to be used in a biorefinery to produce a fermentation product. The method comprises the steps of preparing the biomass into prepared biomass; and pre-treating the prepared biomass into pre-treated biomass by application of a dilute acid having a concentration of about 0.8 to 1.1 percent by weight at a temperature of about 130 to about 170 degrees Celsius for a period of time in a range of about 8 to 12 minutes. The fermentation product can be obtained by separating the pre-treated biomass into a liquid component comprising xylose and a solids component from which glucose can be made available and accessing xylose for fermentation into the fermentation product. The biomass comprises lignocellulosic material. The lignocellulosic material comprises at least one of corn cobs, corn plant husks, corn plant leaves and corn plant stalks.

The present invention also relates to a method to pre-treat biomass to be used in a biorefinery to produce a fermentation product. The method comprises the preparing the biomass into prepared biomass; and pre-treating the prepared biomass into pre-treated biomass by application of a dilute acid having a concentration of about 0.8 to 1.1 percent by weight at a temperature of about 130 to about 170 degrees Celsius for a period of time in a range of about 5 to 15 minutes. The fermentation product can be obtained by separating the pre-treated biomass into a liquid component comprising xylose and a solids component from which glucose can be made available and accessing xylose for fermentation into the fermentation product. The biomass comprises lignocellulosic material. The lignocellulosic material consists essentially of corn cobs, corn plant husks, corn plant leaves and corn plant stalks.

The present invention further relates to a system for pre-treating biomass to be used in a biorefinery to produce a fermentation product. The system comprises an apparatus to preparing the biomass into prepared biomass; an apparatus for pre-treating the prepared biomass into pre-treated biomass by application of a dilute acid having a concentration of about 0.8 to 1.1 percent by weight at a temperature of about 130 to about 170 degrees Celsius for a period of time in a range of about 5 to 15 minutes; an apparatus for separating the pre-treated biomass into a liquid component comprising pentose available to be fermented and a solids component comprising cellulose from which hexose can be made available for fermentation and lignin; a fermentation system configured to produce the fermentation product; and a distillation system configured to recover ethanol from the fermentation product. The fermentation product comprises ethanol produced by fermentation of pentose and ethanol produced from the fermentation of hexose. The biomass comprises lignocellulosic material. The lignocellulosic material consists essentially of corn cobs, corn plant husks, corn plant leaves and corn plant stalks.

The present invention further relates to a method to pre-treat biomass to be used in a biorefinery to produce a fermentation product. The method comprises the steps of preparing the biomass into prepared biomass; and pre-treating the prepared biomass into pre-treated biomass by application of a dilute acid having a concentration of about 0.8 to 1.1 percent by weight at a temperature of about 130 to about 170 degrees Celsius for a period of time in a range of about 8 to 12 minutes. The fermentation product can be obtained by separating the pre-treated biomass into a liquid component comprising xylose and a solids component from which glucose can be made available and accessing xylose for fermentation into the fermentation product. The biomass comprises lignocellulosic material comprising corn cobs, corn plant husks, corn plant leaves and corn stalks and glucan at about 35 to 45 percent by weight and xylan at about 20 to 35 percent by weight. The prepared biomass comprises at least 10 percent solids by weight before the step of applying a dilute acid. The liquid component comprises xylose in a percentage of at least 80 percent of the xylan in the lignocellulosic material. The solids component comprises glucan at about 45 to 65 percent by weight and lignin at about 20 to 30 percent by weight. The solids component comprises at least 75 percent of the glucan from which glucose can be made available in the lignocellulosic material.

### BRIEF DESCRIPTION OF THE DRAWINGS AND TABLES

FIG. 1A is a perspective view of a biorefinery comprising a cellulosic ethanol production facility.

FIG. 1B is a perspective view of a biorefinery comprising a cellulosic ethanol production facility and a corn-based ethanol production facility.

FIG. 2 is a schematic diagram of a system for receipt and preparation of biomass for a cellulosic ethanol production facility.

FIG. 3 is a schematic block diagram of a system for the production of ethanol from biomass.

FIGS. 4A, 4B and 4C are schematic block diagrams of systems for treatment and processing of components from the production of ethanol from biomass.

FIGS. 5A and 5B are schematic diagrams of the process flow for systems for the production of ethanol from biomass.

FIG. 6A is a schematic block diagram of apparatus used for preparation, pre-treatment and separation of biomass.

FIG. 6B is a perspective view of apparatus used to pre-treat and separate the biomass.

FIG. 7A is a schematic block diagram of an exemplary embodiment of an apparatus used for preparation, pre-treatment and separation of biomass.

FIG. 7B is a perspective view of an exemplary embodiment of an apparatus used to pre-treat and separate the biomass.

FIGS. 8A through 8D are diagrams of the operating conditions for the pre-treatment system according to an exemplary embodiment.

FIGS. 9A through 9D are graphs of the results of use of the pre-treatment system according to an exemplary embodiment.

TABLES 1A and 1B list the composition of biomass comprising lignocellulosic plant material from the corn plant according to exemplary and representative embodiments.

TABLES 2A and 2B list the composition of the liquid component of pre-treated biomass according to exemplary and representative embodiments.

TABLES 3A and 3B list the composition of the solids component of pre-treated biomass according to exemplary and representative embodiments.

TABLE 4A lists the composition of prepared biomass supplied to the pre-treatment system according to an exemplary embodiment.

TABLE 4B lists the composition of pre-treated biomass from the pre-treatment system according to an exemplary embodiment.

TABLES 5A and 5B list operating conditions and compositional data from the use of the pre-treatment system according to an exemplary embodiment.

TABLES 6A and 6B list the results of use of the pre-treatment system according to an exemplary embodiment.

TABLE 6C lists the results of use of the pre-treatment system according to an exemplary embodiment.

### DETAILED DESCRIPTION

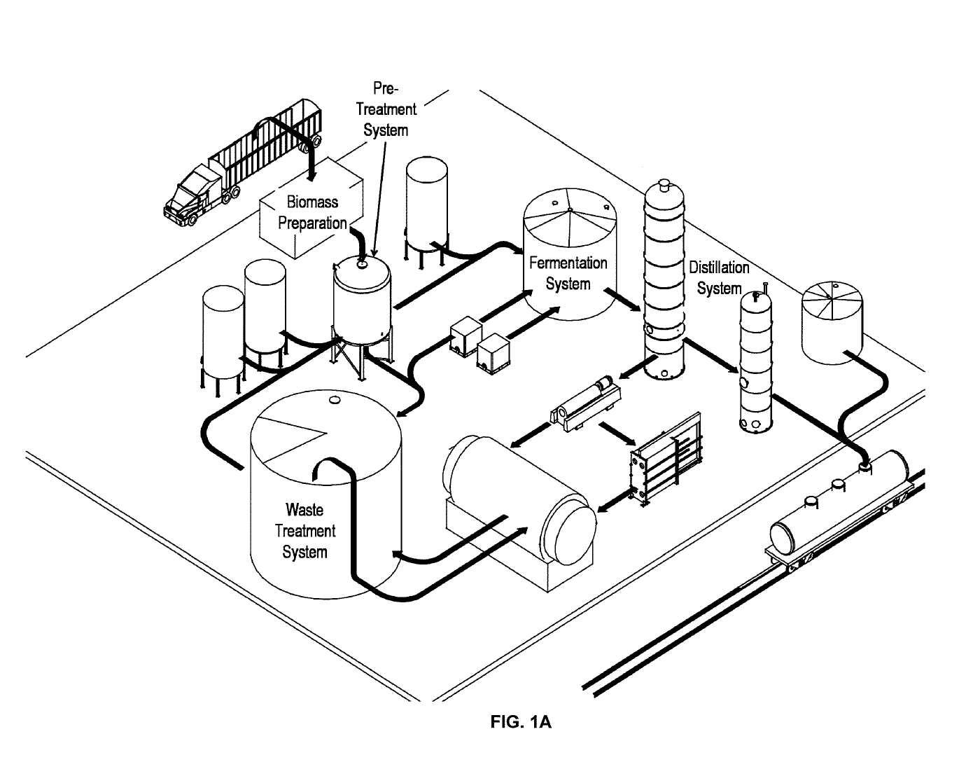

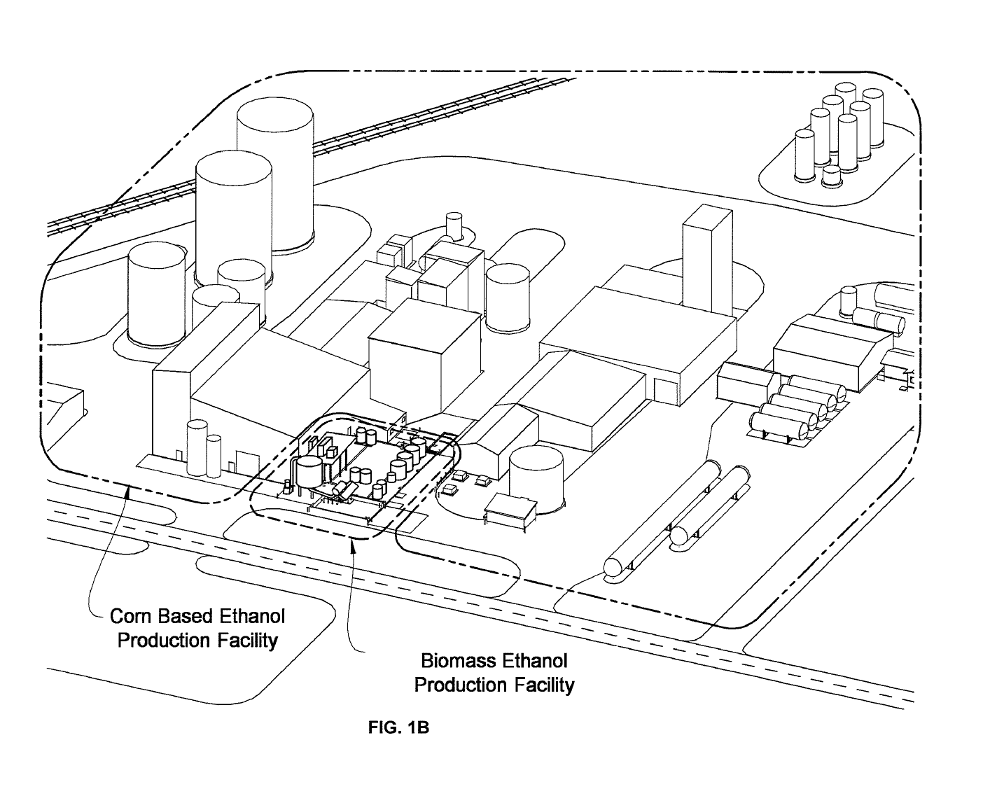

Referring to FIG. 1A, a biorefinery configured to produce ethanol from biomass is shown.

According to an exemplary embodiment, the biorefinery is configured to produce ethanol from biomass in the form of a lignocellulosic feedstock such as plant material from the corn plant (e.g. corn cobs and corn stover). Lignocellulosic feedstock such as lignocellulosic material from the corn plant comprises cellulose (from which C6 sugars such as glucose can be made available) and/or hemicellulose (from which C5 sugars such as xylose and arabinose can be made available).

As shown in FIG. 1A, the biorefinery comprises an area where biomass is delivered and prepared to be supplied to the cellulosic ethanol production facility. The cellulosic ethanol production facility comprises apparatus for preparation, pre-treatment and treatment of the biomass into treated biomass suitable for fermentation into fermentation product in a fermentation system. The facility comprises a distillation system in which the fermentation product is distilled and dehydrated into ethanol. As shown in FIG. 1A, the biorefinery may also comprise a waste treatment system (shown as comprising an anaerobic digester and a generator). According to other alternative embodiments, the waste treatment system may comprise other equipment configured to treat, process and recover components from the cellulosic ethanol production process, such as a solid/waste fuel boiler, anaerobic digester, aerobic digester or other biochemical or chemical reactors.

As shown in FIG. 1B, according to an exemplary embodiment, a biorefinery may comprise a cellulosic ethanol production facility (which produces ethanol from lignocellulosic material and components of the corn plant) co-located with a corn-based ethanol production facility (which produces ethanol from starch contained in the endosperm component of the corn kernel). As indicated in FIG. 1B, by co-locating the two ethanol production facilities, certain plant systems may be shared, for example, systems for dehydration, storage, denaturing and transportation of ethanol, energy/fuel-to-energy generation systems, plant management and control systems, and other systems. Corn fiber (a component of the corn kernel), which can be made available when the corn kernel is prepared for milling (e.g. by fractionation) in the corn-based ethanol production facility, may be supplied to the cellulosic ethanol production facility as a feedstock. Fuel or energy sources such as methane or lignin from the cellulosic ethanol production facility may be used to supply power to either or both co-located facilities. According to other alternative embodiments, a biorefinery (e.g. a cellulosic ethanol production facility) may be co-located with other types of plants and facilities, for example an electric power plant, a waste treatment facility, a lumber mill, a paper plant or a facility that processes agricultural products.

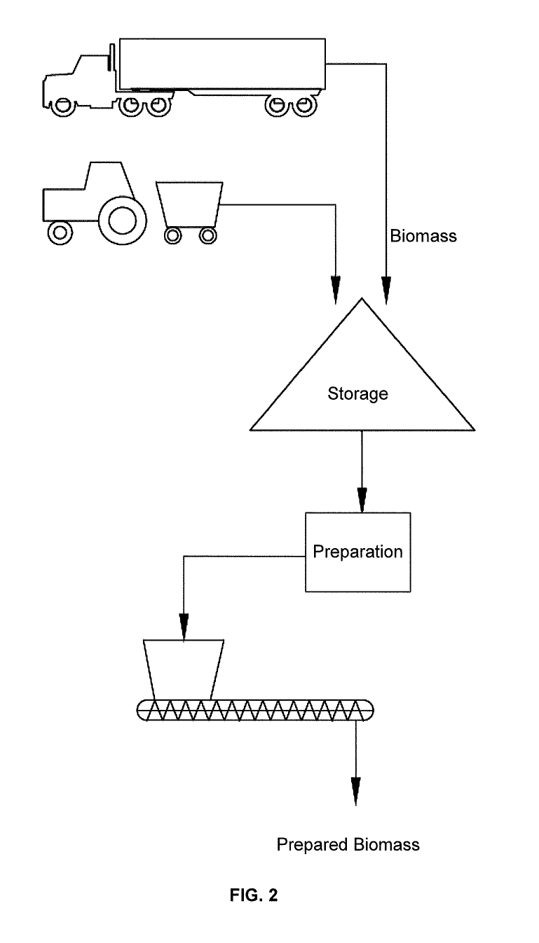

Referring to FIG. 2, a system for preparation of biomass delivered to the biorefinery is shown. The biomass preparation system may comprise apparatus for receipt/unloading of the biomass, cleaning (i.e. removal of foreign matter), grinding (i.e. milling, reduction or densification), and transport and conveyance for processing at the plant. According to an exemplary embodiment, biomass in the form of corn cobs and stover may be delivered to the biorefinery and stored (e.g. in bales, piles or bins, etc.) and managed for use at the facility. According to a preferred embodiment, the biomass may comprise at least 20 to 30 percent corn cobs (by weight) with corn stover and other matter. According to other exemplary embodiments, the preparation system of the biorefinery may be configured to prepare any of a wide variety of types of biomass (i.e. plant material) for treatment and processing into ethanol and other bioproducts at the plant.

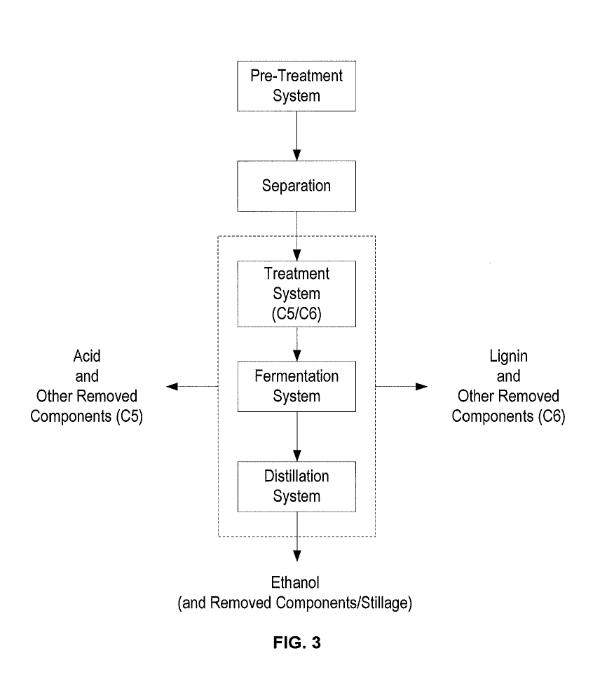

Referring to FIG. 3, a schematic diagram of the cellulosic ethanol production facility is shown. According to a preferred embodiment, biomass comprising plant material from the corn plant is prepared and cleaned at a preparation system. After preparation, the biomass is mixed with water into a slurry and is pre-treated at a pre-treatment system. In the pre-treatment system, the biomass is broken down (e.g. by hydrolysis) to facilitate separation into a liquid component (e.g. a stream comprising the C5 sugars) and a solids component (e.g. a stream comprising cellulose from which the C6 sugars can be made available). The C5-sugar-containing liquid component (C5 stream) and C6-sugar-containing solids component (C6 stream) can be treated (as may be suitable) and fermented in a fermentation system. Fermentation product from the fermentation system is supplied to a distillation system where the ethanol is recovered.

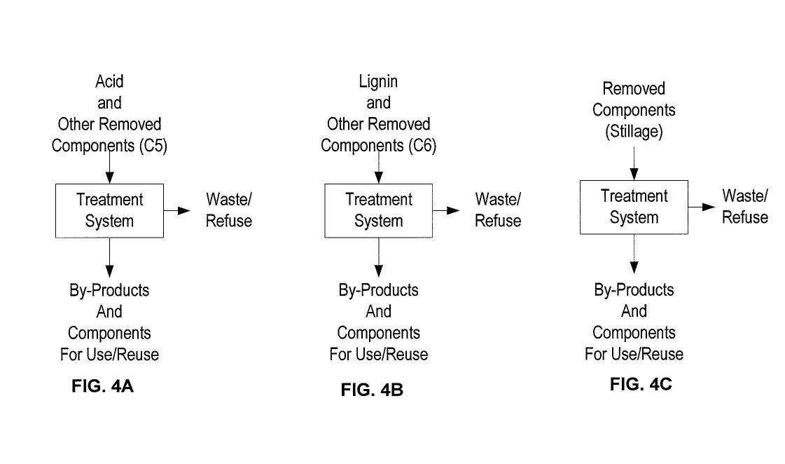

As shown in FIGS. 3 and 4A, removed components from treatment of the C5 stream can be treated or processed to recover by-products, such as organic acids and furfural. As shown in FIGS. 3 and 4B, removed components from treatment of the C6 stream, such as lignin or other components, can be treated or processed into bioproducts or into fuel (such as lignin for a solid fuel boiler or methane produced by treatment of residual/removed matter such as acids and lignin in an anaerobic digester). As shown in FIGS. 4A, 4B and 4C, components removed during treatment and production of ethanol from the biomass from either or both the C5 stream and the C6 stream (or at distillation) may be processed into bioproducts (e.g. by-products or co-products) or recovered for use or reuse. As shown in FIG. 4C, removed components from the distillation system (such as stillage or removed solids) or from the treatment of the fermentation product before distillation (e.g. removed solids and particulate matter, which may comprise residual lignin, etc.) can be treated or processed into bioproducts or fuel (e.g. methane produced in an anaerobic digester).

According to a preferred embodiment, the biomass comprises plant material from the corn plant, such as corn cobs, husks and leaves and stalks; the composition of the plant material (i.e. cellulose, hemicellulose and lignin) will be approximately as indicated in TABLES 1A and 1B. According to a preferred embodiment, the plant material comprises corn cobs, husks/leaves and stalks (i.e. after cleaning/removal of foreign matter); for Example, the plant material may comprise (by weight) up to 100 percent cobs, up to 100 percent husks/leaves, approximately 50 percent cobs and approximately 50 percent husks/leaves, approximately 30 percent cobs and approximately 50 percent husks/leaves and approximately 20 percent stalks, or any other combinations of cobs, husks/leaves and stalks from the corn plant. See TABLE 1A. According to an exemplary embodiment, corn stalks comprise the upper half or three-quarters portion of the stalk. According to an alternative embodiment, the lignocellulosic plant material may comprise fiber from the corn kernel (e.g. in some combination with other plant material). TABLE 1B provides typical and expected ranges believed to be representative of the composition of biomass comprising lignocellulosic material from the corn plant. According to exemplary embodiments, the lignocellulosic plant material of the biomass (from the corn plant) will comprise (by weight) cellulose at about 30 to 55 percent, hemicellulose at about 20 to 50 percent, and lignin at about 10 to 25 percent; according to a particularly preferred embodiment, the lignocellulosic plant material of the biomass (cobs, husks/leaves and stalk portions from the corn plant) will comprise (by weight) cellulose at about 35 to 45 percent, hemicellulose at about 24 to 42 percent, and lignin at about 12 to 20 percent. According to a particularly preferred embodiment, pre-treatment of the biomass will yield a liquid component that comprises (by weight) xylose at no less than 1.0 percent and a solids component that comprises (by weight) cellulose (from which glucose can be made available) at no less than 45 percent.

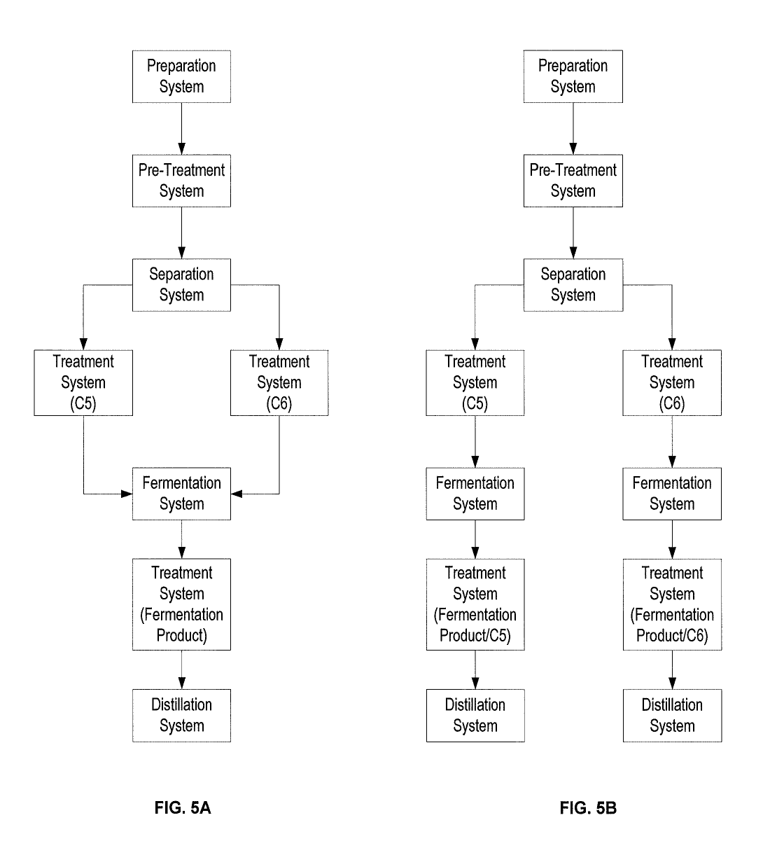

Referring to FIGS. 5A and 5B, exemplary embodiments of systems for the production of ethanol from biomass are shown. As shown in FIGS. 5A and 5B, biomass is pre-treated in a pre-treatment system and then separated into a liquid component and a solids component.

According to a preferred embodiment, in the pre-treatment system an acid will be applied to the prepared biomass to facilitate the break down of the biomass for separation into the liquid component (C5 stream from which fermentable C5 sugars can be recovered) and the solids component (C6 stream from which fermentable C6 sugars can be accessed). According to a preferred embodiment, the acid can be applied to the biomass in a reaction vessel under determined operating conditions (i.e. acid concentration, pH, temperature, time, pressure, solids loading, flow rate, supply of process water or steam, etc.) and the biomass can be agitated/mixed in the reaction vessel to facilitate the break down of the biomass. According to exemplary embodiments, an acid such as sulfuric acid, hydrochloric acid, nitric acid, phosphoric acid, acetic acid, etc. (or a formulation/mixture of acids) can be applied to the biomass. According to a particularly preferred embodiment, sulfuric acid will be applied to the biomass in pre-treatment.

The liquid component (C5 stream) comprises water, dissolved sugars (such as xylose, arabinose and glucose) to be made available for fermentation into ethanol, acids and other soluble components recovered from the hemicellulose. (TABLE 2B provides typical and expected ranges believed to be representative of the composition of biomass comprising lignocellulosic material from the corn plant.) According to an exemplary embodiment, the liquid component may comprise approximately 5 to 7 percent solids (i.e. suspended/residual solids such as partially-hydrolyzed hemicellulose, cellulose and lignin). According to a particularly preferred embodiment, the liquid component will comprise at least 2 to 6 percent xylose (by weight); according to other exemplary embodiments, the liquid component will comprise no less than 1 to 2 percent xylose (by weight). According to a preferred embodiment, the liquid component may comprise xylose in a percentage of at least 50 percent of the hemicellulose in the lignocellulosic material. In a particularly preferred embodiment, the liquid component comprises xylose in a percentage of at least 70 percent of the xylan in the lignocellulosic material. TABLES 2A and 2B list the composition of the liquid component of pre-treated biomass (from prepared biomass as indicated in TABLES 1A and 1B) according to exemplary and representative embodiments.

The solids component (C6 stream) comprises water, acids and solids such as cellulose from which sugar, such as glucose, can be made available for fermentation into ethanol, and lignin. (TABLE 3B provides typical and expected ranges believed to be representative of the composition of biomass comprising lignocellulosic material from the corn plant.) According to an exemplary embodiment, the solids component may comprise approximately 10 to 40 percent solids (by weight) (after separation); according to a particularly preferred embodiment, the solids component will comprise approximately 20 to 30 percent solids (by weight). According to a preferred embodiment, the solids in the solids component comprise no less than 30 percent cellulose and the solids component may also comprise other dissolved sugars (e.g. glucose and xylose). TABLES 3A and 3B list the composition of the solids component of pre-treated biomass (from prepared biomass as indicated in TABLES 1A and 1B) according to exemplary and representative embodiments.

During pre-treatment, the severity of operating conditions (such as pH, temperature and time) may cause formation of components that are inhibitory to fermentation. For example, under some conditions, the dehydration of C5 sugars (such as xylose or arabinose) may cause the formation of furfural and/or hydroxymethylfurfural (HMF). Acetic acid may also be formed, for example when acetate is released during the break down of cellulose in pre-treatment. Sulfuric acid, which may be added to prepared biomass to facilitate pre-treatment, if not removed or neutralized, may also be inhibitory to fermentation. According to an exemplary embodiment, by adjusting pre-treatment conditions (such as pH, temperature and time), the formation of inhibitors can be reduced or managed; according to other exemplary embodiments, components of the pre-treated biomass may be given further treatment to remove or reduce the level of inhibitors (or other undesirable matter).

Referring to FIGS. 5A and 5B, after pre-treatment and separation the C5 stream and the C6 stream are processed separately; as shown, the C5 stream and the C6 stream may be processed separately prior to co-fermentation (C5/C6 fermentation as shown in FIG. 5A) or processed separately including separate fermentation (separate C5 fermentation and C6 fermentation as shown in FIG. 5B).

Treatment of the C5 stream (liquid component) of the biomass may be performed in an effort to remove components that are inhibitory to efficient fermentation (e.g. furfural, HMF, sulfuric acid and acetic acid) and residual lignin (or other matter) that may not be fermentable from the C5 sugar component so that the sugars (e.g. xylose, arabinose, as well as other sugars such as glucose) are available for fermentation. The C5 sugars in the C5 stream may also be concentrated to improve the efficiency of fermentation (e.g. to improve the titer of ethanol for distillation).

Treatment of the C6 stream (solids component) of the biomass may be performed to make the C6 sugars available for fermentation. According to a preferred embodiment, hydrolysis (such as enzyme hydrolysis) may be performed to access the C6 sugars in the cellulose; treatment may also be performed in an effort to remove lignin and other non-fermentable components in the C6 stream (or to remove components such as residual acid or acids that may be inhibitory to efficient fermentation).

According to an exemplary embodiment shown in FIG. 5A, after pre-treatment and separation the C5 stream and the C6 stream can be treated separately and subsequently combined after treatment (e.g. as a slurry) for co-fermentation in the fermentation system to produce a C5/C6 fermentation product from the available sugars (e.g. xylose and glucose); the C5/C6 fermentation product can (after treatment, if any) be supplied to the distillation system for recovery of the ethanol (e.g. through distillation and dehydration). According to an exemplary embodiment shown in FIG. 5B, the C5 stream and the C6 stream can each be separately processed through fermentation and distillation (after treatment, if any) to produce ethanol. According to any preferred embodiment, a suitable fermenting organism (ethanologen) will be used in the fermentation system; the selection of an ethanologen may be based on various considerations, such as the predominant types of sugars present in the slurry. Dehydration and/or denaturing of the ethanol produced from the C5 stream and the C6 stream may be performed either separately or in combination.

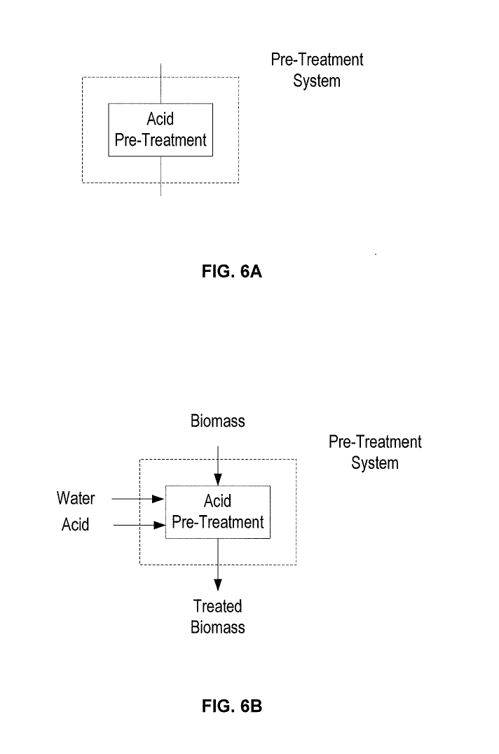

Referring to FIGS. 6A and 6B, the process flow for the pre-treatment system is shown. Prepared biomass is supplied to the pre-treatment system along with water and acid. The pre-treatment system comprises a reaction vessel containing the biomass, water and acid under pre-determined operating conditions, namely acid concentration, reaction time and temperature.

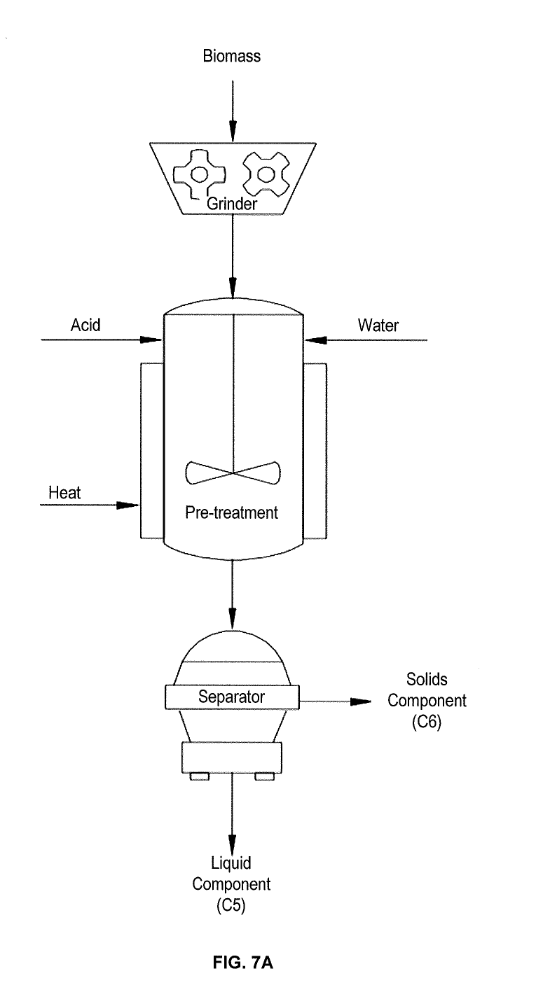

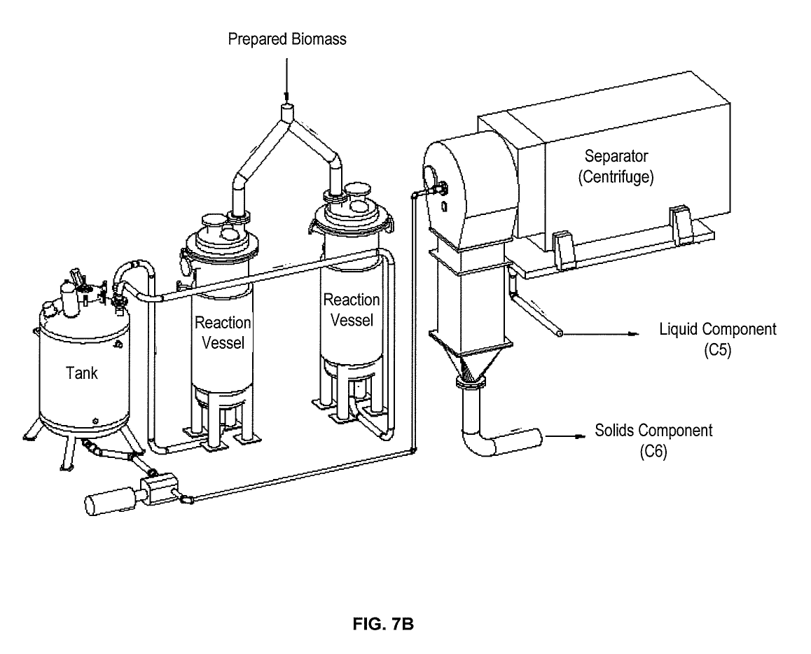

FIGS. 7A and 7B show the apparatus used for preparation, pre-treatment and separation of lignocellulosic biomass according to an exemplary embodiment. As shown, biomass is prepared in a grinder (e.g. grinder or other suitable apparatus or mill). According to an exemplary embodiment, pre-treatment is performed in at least one reaction vessel supplied with prepared biomass and acid/water in a predetermined concentration (or pH). According to an alternative embodiment, pre-treatment is performed in a plurality of reaction vessels. As shown in FIG. 7A, the pre-treated biomass can be separated in a centrifuge into a liquid component (e.g. a C5 stream comprising primarily liquids with some solids and which may be referred to as “pentose liquor”) and a solids component (e.g. a C6 stream comprising liquids and solids such as lignin and cellulose from which glucose can be made available by further treatment).

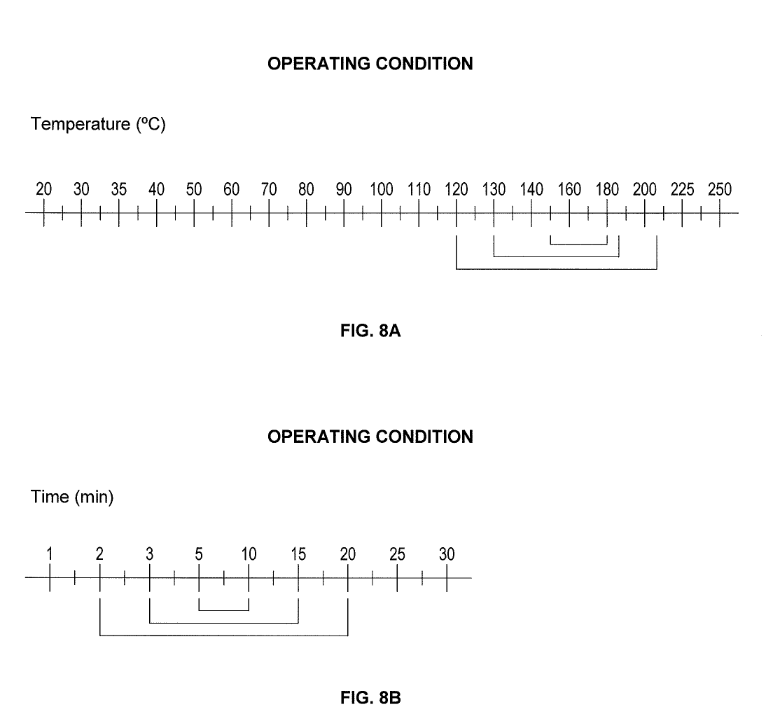

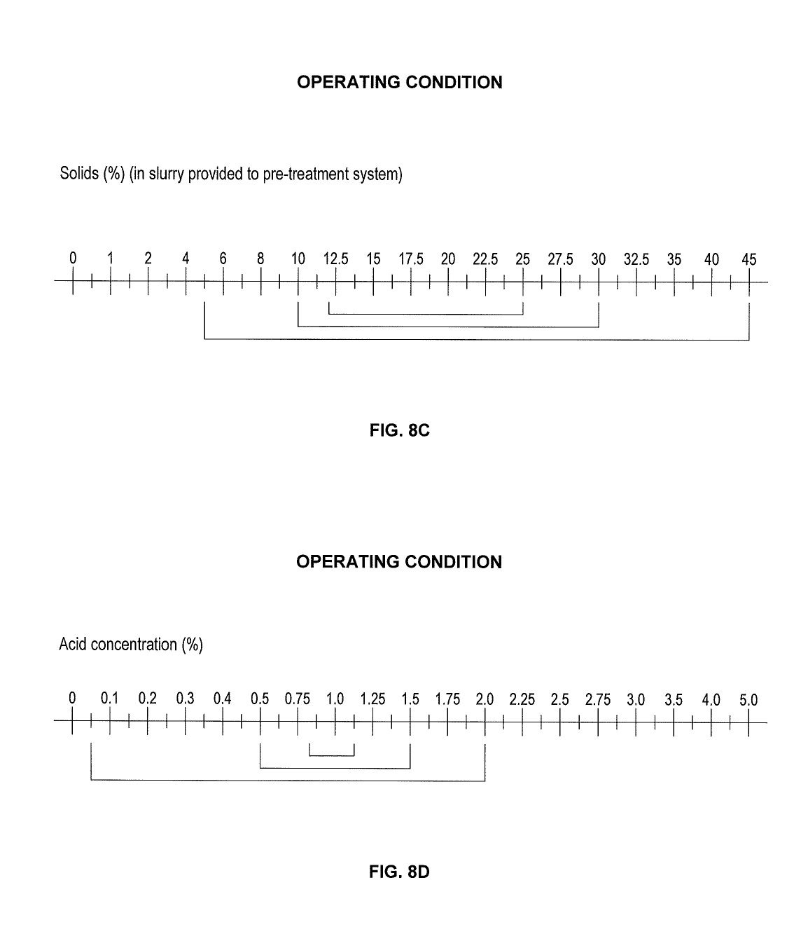

FIGS. 8A through 8D show operating conditions for subject conditions or parameters for the pre-treatment of biomass according to an exemplary embodiment. Operating conditions are shown in the form of nested ranges comprising an acceptable operating range (the outer/wide range shown), a preferred operating range (the middle range shown), and a particularly preferred operating range (the inner/narrow range shown) for each subject condition or parameter.

FIG. 8A shows the temperature ranges for operation the pre-treatment system. According to an exemplary embodiment, the operating temperature range for pre-treating biomass is about 120 to about 210 degrees Celsius. According to a preferred embodiment, the operating temperature is from about 130 to about 185 degrees Celsius. According to a particularly preferred embodiment, the operating temperature is from about 150 to about 180 degrees Celsius.

FIG. 8B shows the reaction time for operation of the pre-treatment system (in percent, by weight). According to an exemplary embodiment, the pre-treatment time is from about 2 to 20 minutes. According to a preferred embodiment, the pre-treatment time is from about 3 to about 15 minutes. According to a particularly preferred embodiment, the pre-treatment time is from about 8 to about 12 minutes.

FIG. 8C shows the solids loading for operation of the pre-treatment system (in percent, by weight). According to an exemplary embodiment, the solids loading is from about 5 to about 45 percent. Acceding to a preferred embodiment, the solids loading is from about to about 30 percent. According to a particularly preferred embodiment, the solids loading is from about 12 to about 25 percent solids.

FIG. 8D shows the acid concentration (sulfuric acid in water) for operation of the pre-treatment system (in percent, by weight). According to an exemplary embodiment, the acid concentration is from about 0.05 to about 2.0 percent sulfuric acid in the pre-treatment solution. According to a preferred embodiment, the acid concentration is from about 0.5 to about 1.5 percent. According to a particularly preferred embodiment, the acid concentration is from about 0.8 to about 1.1 percent.

According to a preferred embodiment, pre-treatment is conducted in a closed reaction vessel and at a pressure that may increase during the reaction from ambient pressure to approximately 100-120 pounds per square inch.

A series of examples were conducted according to an exemplary embodiment of the system in an effort to evaluate the efficiency of pre-treatment of biomass by dilute acid. Data from the examples is shown in TABLES 4A and 4B (composition of prepared biomass and pre-treated biomass) and 5A and 5B (operating conditions and compositional data for samples). The system used for the examples comprised a temperature-controlled reaction vessel. The composition of a sample of prepared biomass and pre-treated biomass is shown in TABLE 1A (prepared biomass) and 1B (pre-treated biomass). Samples were prepared from biomass comprising lignocellulosic material from the corn plant (i.e. corn cobs, husks/leaves, stalks) as represented in TABLE 1.

### Example 1

The system was used in Example 1 to determine the yield of xylose and level of furfural (inhibitor) from pre-treated biomass. The prepared biomass was pre-treated in a reaction vessel and separated into a liquid component and a solids component (as indicated in FIG. 7A). Prepared biomass was loaded into the reaction vessel with water and sulfuric acid. At the start of pre-treatment, the reaction vessel contained (by weight) about 14.3 percent solids (e.g. from the prepared biomass) and about 0.9 percent sulfuric acid (added, with water). Pre-treatment was conducted at about 150 degrees Celsius for a time of about 5 minutes. After pre-treatment and separation, the liquid component of the pre-treated biomass was analyzed. The concentration of xylose in the liquid component was approximately 4.6 percent (by weight). It was observed that under the operating conditions a yield of about 85 to over 95 percent of xylose from the xylan in the prepared biomass (a xylose concentration of up to about 4.6 percent by weight in the liquid component) along with glucose (at a concentration of about 0.44 percent by weight in the liquid component) and a relatively low level of furfural (an inhibitor present at a concentration of about 570 PPM). The results are shown in TABLES 4A and 4B.

### Example 2

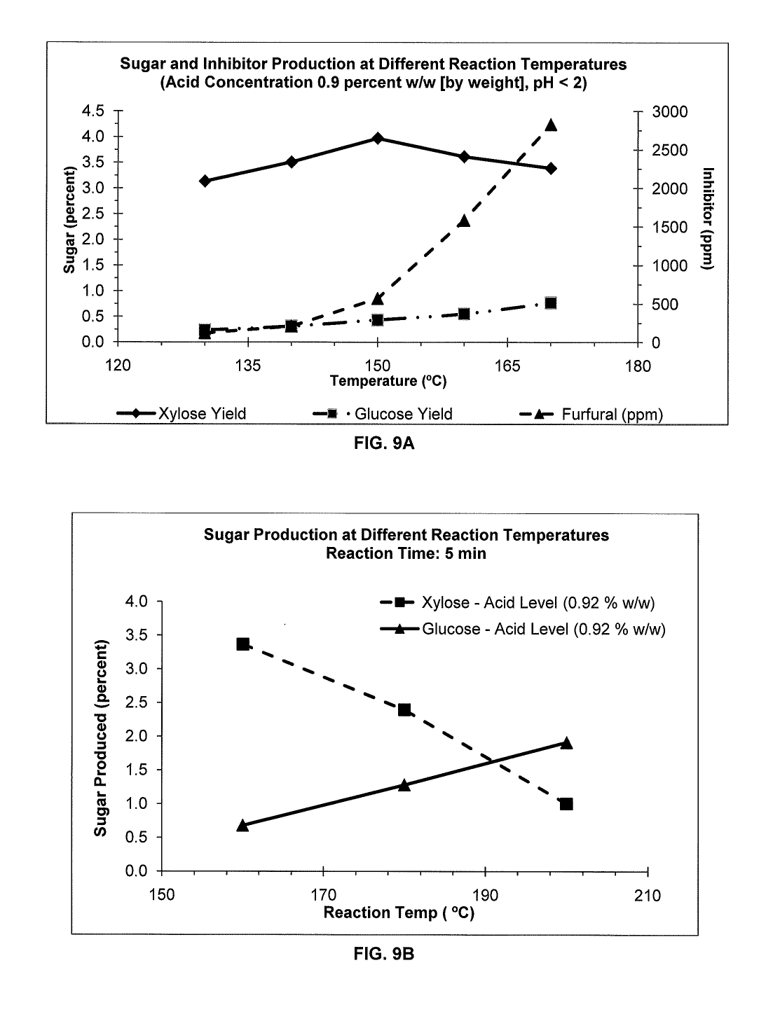

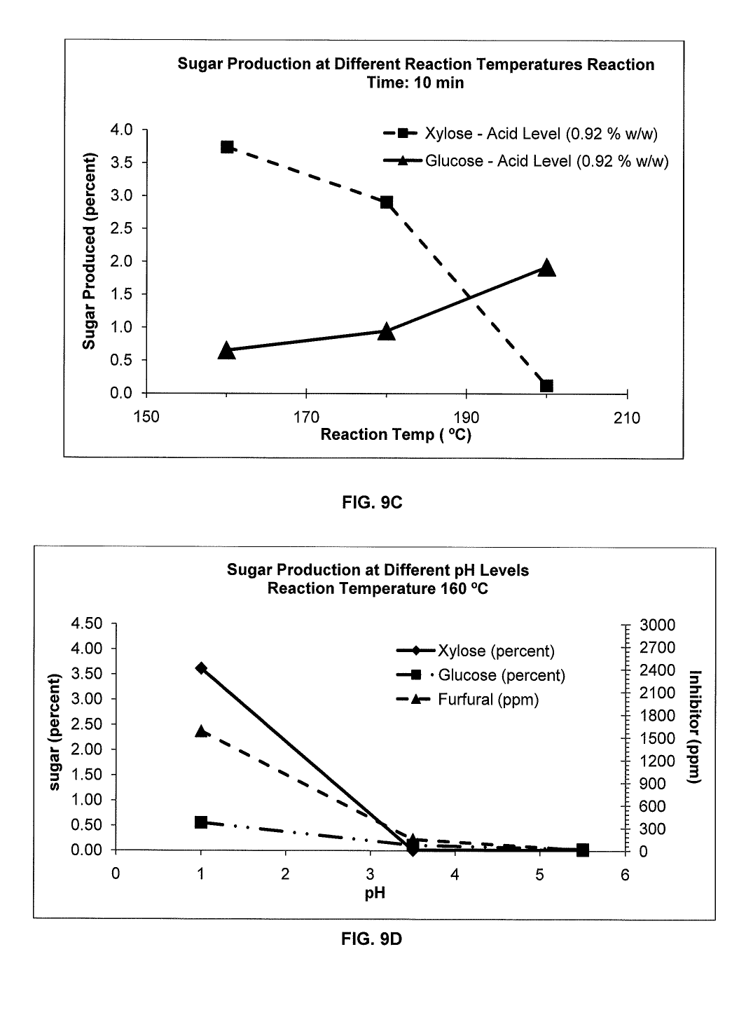

The system was used in Example 2 to determine the effect of reaction temperature on the sugar (xylose and glucose) yield and inhibitor (furfural) levels from prepared biomass after pre-treatment. Prepared biomass was loaded into the reaction vessel with water and sulfuric acid. At the start of pre-treatment, the reaction vessel contained (by weight) about 14.3 percent solids (e.g. from the prepared biomass) and about 0.9 percent by weight sulfuric acid (added, with water). Pre-treatment was conducted at a temperature range of about 130 to about 170 degrees Celsius and for a time of about 10 minutes. After pre-treatment and separation, the liquid component of the pre-treated biomass was analyzed. It was observed that under the operating conditions the yield of xylose from xylan in the prepared biomass (a xylose concentration of about 4 percent by weight in the liquid component) could be maximized at about 150 degrees Celsius. The results are shown in FIG. 9A and in TABLE 5B.

### Example 3

The system was used in Example 3 to determine the effects of reaction temperature and reaction time on sugar (xylose and glucose) yield and inhibitor (furfural) levels from prepared biomass after pre-treatment. Prepared biomass was loaded into the reaction vessel with water and sulfuric acid. At the start of pre-treatment, the reaction vessel contained (by weight) about 14.3 percent solids (e.g. from the prepared biomass) and about 0.9 percent sulfuric acid (added, with water). Pre-treatment was conducted at temperatures in a range of about 160 to about 200 degrees Celsius and for times of about 5 minutes and about 10 minutes. After pre-treatment and separation, the liquid component of the pre-treated biomass was analyzed. It was observed that under the operating conditions the yield of xylose from xylan in the prepared biomass (a xylose concentration of about 3.7 percent by weight in the liquid component) could be maximized at about 160 degrees Celsius and a time of about 10 minutes. The results are shown in FIG. 9B (5 minute reaction time), FIG. 9C (10 minute reaction time), TABLE 6A and TABLE 6B.

### Example 4

The system was used in Example 4 to determine the effect of acidity (i.e. pH/acid concentration) on sugar (xylose and glucose) yield and inhibitor (furfural) levels from prepared biomass after pre-treatment. Prepared biomass was loaded into the reaction vessel with water and sulfuric acid. At the start of pre-treatment, the reaction vessel contained (by weight) about 14.3 percent solids (e.g. from the prepared biomass) and with acid concentration of pH 1, pH 3.5 and pH 5.5. Pre-treatment was conducted at a temperature of about 160 degrees Celsius and for a time of about 10 minutes. After pre-treatment and separation, the liquid component of the pre-treated biomass was analyzed. It was observed that under the operating conditions the yield of xylose and glucose in the prepared biomass (a xylose concentration of about 3.6 percent and a glucose concentration of about 0.56 percent by weight in the liquid component) could generally be optimized and a suitably low level of furfural (an inhibitor) (at a concentration of about 1600 PPM) would be present at a pH of about 1. The results are shown in FIG. 9D and TABLE 6C.

The embodiments as disclosed and described in the application (including the FIGURES and Examples) are intended to be illustrative and explanatory of the present inventions. Modifications and variations of the disclosed embodiments, for example, of the apparatus and processes employed (or to be employed) as well as of the compositions and treatments used (or to be used), are possible; all such modifications and variations are intended to be within the scope of the present inventions.

TABLE 1A
Biomass Composition

| | | | Cellulose | Hemicellulose | | | | | |
|---|---|---|---|---|---|---|---|---|---|
| Cob | Husks/Leaves | Stalk | (Glucan) | Xylan | Arabinan | Acetate | Composite | Lignin | Ash |
| (percent) | (percent) | (percent) | (percent) | (percent) | (percent) | (percent) | (percent) | (percent) | (percent) |
| 100 | 0 | 0 | 36.0 | 33.3 | 3.6 | 3.0 | 39.9 | 14.9 | 2.2 |
| 0 | 100 | 0 | 37.2 | 25.6 | 4.9 | 2.2 | 32.7 | 13.0 | 7.7 |
| 0 | 0 | 100 | 41.7 | 22.5 | 2.4 | 2.6 | 27.5 | 18.3 | 3.7 |
| 50 | 0 | 50 | 38.8 | 27.9 | 3.0 | 2.8 | 33.7 | 16.6 | 3.0 |
| 50 | 50 | 0 | 36.6 | 29.5 | 4.2 | 2.6 | 36.3 | 14.0 | 5.0 |
| 30 | 50 | 20 | 37.7 | 27.3 | 4.0 | 2.5 | 33.8 | 14.6 | 5.3 |

TABLE 1B
Biomass Typical and Expected Composition

| | Cellulose | | | |
|---|---|---|---|---|
| | (Glucan) | Hemicellulose | Lignin | Ash |
| | (percent) | (percent) | (percent) | (percent) |
| | (approx.) | (approx.) | (approx.) | (approx.) |
| Typical Range | 35-45 | 24-42 | 12-20 | 2-8 |
| Expected Range | 30-55 | 20-50 | 10-25 | 1-10 |

TABLE 2A
Pre-Treated Biomass Liquid Component Composition

| Cob | Husks/Leaves | Stalk | Glucose | Xylose | Arabinose | Acetic Acid |
|---|---|---|---|---|---|---|
| (percent) | (percent) | (percent) | (percent) | (percent) | (percent) | (ppm) |
| 100 | 0 | 0 | 0.4 | 4.8 | 0.5 | 6090 |
| 0 | 100 | 0 | 0.4 | 2.7 | 0.5 | 3400 |
| 0 | 0 | 100 | 0.4 | 4.2 | 0.4 | 6180 |
| 50 | 0 | 50 | 0.4 | 4.5 | 0.4 | 6135 |
| 30 | 50 | 20 | 0.4 | 3.6 | 0.5 | 4763 |

TABLE 2B
Pre-Treated Biomass Liquid Component
Typical and Expected Composition

| | Glucose | Xylose | Arabinose | Acetic Acid |
|---|---|---|---|---|
| | (percent) | (percent) | (percent) | (ppm) |
| | (approx.) | (approx.) | (approx.) | (approx.) |
| Typical Range | 0-1 | 2-6 | 0-1 | 3000-6400 |
| Expected Range | 0-1 | 1-8 | 0-1 | 2000-8000 |

TABLE 3A
Pre-Treated Biomass Solids Component Composition

| | | | Cellulose | Hemicellulose | | | | | |
|---|---|---|---|---|---|---|---|---|---|
| Cob | Husks/Leaves | Stalk | (Glucan) | Xylan | Arabinan | Acetate | Composite | Lignin | Ash |
| (percent) | (percent) | (percent) | (percent) | (percent) | (percent) | (percent) | (percent) | (percent) | (percent) |
| 100 | 0 | 0 | 60.2 | 9.5 | 0.9 | 0.3 | 10.7 | 26.7 | 1.2 |
| 0 | 100 | 0 | 54.4 | 1.3 | 0.7 | 0.7 | 10.4 | 23.8 | 9.7 |
| 0 | 0 | 100 | 51.1 | 1.4 | 1.0 | 1.0 | 15.4 | 27.3 | 3.1 |
| 50 | 0 | 50 | 55.7 | 5.5 | 0.9 | 0.6 | 13.1 | 27.0 | 2.2 |
| 50 | 50 | 0 | 57.3 | 5.4 | 0.8 | 0.5 | 10.6 | 25.2 | 5.4 |
| 30 | 50 | 20 | 55.5 | 3.8 | 0.8 | 0.6 | 11.5 | 25.4 | 5.8 |

TABLE 3B
Pre-Treated Biomass Solids Component
Typical and Expected Composition

| | Cellulose | | | |
|---|---|---|---|---|
| | (Glucan) | Hemicellulose | Lignin | Ash |
| | (percent) | (percent) | (percent) | (percent) |
| | (approx.) | (approx.) | (approx.) | (approx.) |
| Typical Range | 48-62 | 8-17 | 22-30 | 1-10 |
| Expected Range | 45-65 | 5-20 | 20-32 | 1-10 |

TABLE 4A

| Component | Prepared Biomass |
|---|---|
| Lignin | 15.95 ± 0.40 percent |
| Cellulose | 36.83 ± 0.21 percent |
| Hemicellulose | 39.83 percent |
| Xylan (in hemicellulose) | 33.90 ± 0.60 percent (of total solids) |
| Arabinan (in hemicellulose) | 2.83 ± 0.41 percent (of total solids) |
| Acetate (in hemicellulose) | 3.11 ± 0.12 percent (of total solids) |
| Ash and extractives (soluble | 6.44 ± 0.29 percent |
| in water/ethanol) | |

TABLE 4B

| Component | Pre-treated Biomass* |
|---|---|
| xylose (from pre-treatment) | 4.6 percent (85-100 percent yield) |
| furfural | 570 PPM (0.057 percent) |
| glucose | 0.44 percent |

*0.9 percent w/w H₂SO₄, 150° C., 14.3 percent solids, 5 min pre-treatment time

TABLE 5A

| | Solids | Acid Level | Reaction | Reaction |
|---|---|---|---|---|
| | Loading | (percent w/w, | Time | Temp. |
| Sample | (percent) | in solution) | (min) | (° C.) |
| 1 | 14.3 | 0.9 | 10 | 130 |
| 2 | 14.3 | 0.9 | 10 | 140 |
| 3 | 14.3 | 0.9 | 10 | 150 |
| 4 | 14.3 | 0.9 | 10 | 160 |
| 5 | 14.3 | 0.9 | 10 | 170 |

TABLE 5B

| | Reaction Temp | Xylose | Glucose | Furfural |
|---|---|---|---|---|
| Sample | (° C.) | (percent w/v) | (percent w/v) | (ppm) |
| 1 | 130 | 3.1 | 0.24 | 120 |
| 2 | 140 | 3.5 | 0.31 | 210 |
| 3 | 150 | 4.0 | 0.44 | 570 |
| 4 | 160 | 3.6 | 0.56 | 1580 |
| 5 | 170 | 3.4 | 0.77 | 2830 |

TABLE 6A
Reaction Time 5 min

| Temperature | Xylose | Glucose |
|---|---|---|
| (° C.) | (percent) | (percent) |
| 160 | 3.37 | 0.68 |
| 180 | 2.40 | 1.28 |
| 200 | 1.00 | 1.91 |

TABLE 6B
Reaction Time 10 min

| Temperature | Xylose | Glucose |
|---|---|---|
| (° C.) | (percent) | (percent) |
| 160 | 3.74 | 0.66 |
| 180 | 2.91 | 0.95 |
| 200 | 0.12 | 1.92 |

TABLE 6C

| | Xylose | Glucose | Furfural |
|---|---|---|---|
| pH | (percent) | (percent) | (ppm) |
| 1 | 3.61 | 0.56 | 1583 |
| 3.5 | 0.01 | 0.11 | 151 |
| 5.5 | 0.02 | 0.03 | 12 |

We claim:

1. A method to pre-treat biomass to be used in a biorefinery to produce a fermentation product comprising the steps of:
preparing the biomass into prepared biomass; and
pre-treating the prepared biomass into pre-treated biomass by application of a dilute acid having a concentration of about 0.8 to 1.1 percent by weight at a temperature of about 130 to about 170 degrees Celsius for a period of time in a range of about 8 to 12 minutes;
wherein the fermentation product can be obtained by separating the pre-treated biomass into a liquid component comprising xylose and a solids component from which glucose can be made available and accessing xylose for fermentation into the fermentation product;
wherein the biomass comprises lignocellulosic material;
wherein the lignocellulosic material comprises at least one of corn cobs, corn plant husks, corn plant leaves and corn plant stalks.

2. The method of claim 1 wherein the lignocellulosic material comprises cellulose at about 30 to 55 percent by weight and hemicellulose at about 20 to 50 percent by weight.

3. The method of claim 1 wherein the lignocellulosic material (a) comprises corn cobs, corn plant husks, corn plant leaves and corn stalks and (b) comprises cellulose at about 35 to 45 percent by weight and hemicellulose at about 24 to 42 percent by weight.

4. The method of claim 1 wherein the lignocellulosic material consists essentially of corn cobs, corn plant husks, corn plant leaves and corn stalks.

5. The method of claim 1 wherein the lignocellulosic material comprises cellulose at about 30 to 55 percent by weight, hemicellulose at about 20 to 50 percent by weight and lignin at about 10 to 25 percent by weight.

6. The method of claim 1 wherein the lignocellulosic material comprises corn cobs, corn plant husks, corn plant leaves, corn stalks and corn kernel fiber.

7. The method of claim 1 wherein the lignocellulosic material comprises glucan.

8. The method of claim 7 wherein the glucan comprises cellulose.

9. The method of claim 1 wherein the lignocellulosic material comprises xylan.

10. The method of claim 9 wherein the hemicellulose comprises xylan.

11. The method of claim 1 wherein the liquid component comprises xylose at about 2 to 6 percent by weight.

12. The method of claim 1 wherein the liquid component comprises glucose at up to 1 percent by weight.

13. The method of claim 1 wherein the liquid component comprises solids and comprises xylose at about 40 percent by weight of the solids.

14. The method of claim 1 wherein the liquid component comprises xylose in a percentage of at least 50 percent of the hemicellulose in the lignocellulosic material.

15. The method of claim 9 wherein the liquid component comprises xylose in a percentage of at least 70 percent of the xylan in the lignocellulosic material.

16. The method of claim 9 wherein the liquid component comprises xylose in a percentage of at least 80 percent of the xylan in the lignocellulosic material.

17. The method of claim 9 wherein the liquid component comprises xylose in a percentage of at least 90 percent of the xylan in the lignocellulosic material.

18. The method of claim 1 wherein the liquid component comprises furfural.

19. The method of claim 1 wherein the liquid component comprises acetic acid.

20. The method of claim 1 wherein the solids component comprises lignocellulosic material.

21. The method of claim 1 wherein the solids component comprises cellulose at about 45 to 65 percent by weight.

22. The method of claim 1 wherein the solids component comprises lignin at about 20 to 30 percent by weight.

23. The method of claim 1 wherein the solids component comprises at least 75 percent of the cellulose from which glucose can be made available in the lignocellulosic material.

24. The method of claim 1 wherein the liquid component comprises xylose at about 3.5 to 5.2 percent by weight.

25. The method of claim 1 wherein the liquid component comprises xylose at about 4.0 to 5.0 percent by weight.

26. The method of claim 1 wherein the solids component comprises at least 90 percent of the cellulose in the lignocellulosic material.

27. The method of claim 1 wherein the liquid component comprises at least a portion of the acid from the step of applying a dilute acid to the biomass.

28. The method of claim 1 further comprising the step of accessing xylose and glucose for fermentation into the fermentation product.

29. The method of claim 28 further comprising the steps of removing the lignin from the fermentation product; and distilling the fermentation product to recover ethanol.

30. The method of claim 1 wherein the lignocellulosic material comprises plant material and further comprising the step of grinding the plant material into prepared biomass prior to the step of applying a dilute acid.

31. The method of claim 30 wherein the dilute acid is sulfuric acid.

32. The method of claim 30 wherein the prepared biomass comprises at least 10 percent solids by weight in a slurry provided to pre-treatment.

33. The method of claim 30 wherein the dilute acid has a concentration of about 0.85 percent to about 0.95 percent.

34. The method of claim 11 wherein the step of separating the pre-treated biomass comprises the use of a centrifuge.

35. The method of claim 11 further comprising the step of distilling the fermentation product to recover ethanol.

36. The method of claim 30 wherein the liquid component comprises at least a portion of the acid from the step of applying a dilute acid to the biomass.

37. A method to pre-treat biomass to be used in a biorefinery to produce a fermentation product comprising the steps of:
preparing the biomass into prepared biomass; and
pre-treating the prepared biomass into pre-treated biomass by application of a dilute acid having a concentration of about 0.8 to 1.1 percent by weight at a temperature of about 130 to about 170 degrees Celsius for a period of time in a range of about 5 to 15 minutes;
wherein the fermentation product can be obtained by separating the pre-treated biomass into a liquid component comprising xylose and a solids component from which glucose can be made available and accessing xylose for fermentation into the fermentation product;
wherein the biomass comprises lignocellulosic material;
wherein the lignocellulosic material consists essentially of corn cobs, corn plant husks, corn plant leaves and corn plant stalks.

38. The method of claim 37 wherein the step of pre-treating the prepared biomass is performed in a reaction vessel.

39. The method of claim 37 wherein the step of pre-treating the prepared biomass is performed at about 135 to 160 degrees Celsius.

40. The method of claim 37 wherein the step of pre-treating the prepared biomass comprises the application of sulfuric acid having a concentration in a range of about 0.85 to about 1.0 percent by weight within the reaction vessel.

41. The method of claim 37 wherein the prepared biomass supplied to the reaction vessel has a composition of at least 10 percent solids by weight and the liquid component of the biomass after separation comprises xylose.

42. The method of claim 37 wherein the liquid component comprises at least a portion of the acid applied in pre-treating the prepared biomass.

43. The method of claim 37 wherein the dilute acid has a concentration in a range of about 0.85 percent to about 0.95 percent by weight within the reaction vessel.

44. A system for pre-treating biomass to be used in a biorefinery to produce a fermentation product comprising:
an apparatus for preparing the biomass into prepared biomass;
an apparatus for pre-treating the prepared biomass into pre-treated biomass by application of a dilute acid having a concentration of about 0.8 to 1.1 percent by weight at a temperature of about 130 to about 170 degrees Celsius for a period of time in a range of about 5 to 15 minutes;
an apparatus for separating the pre-treated biomass into a liquid component comprising pentose available to be fermented and a solids component comprising cellulose from which hexose can be made available for fermentation and lignin; and
a fermentation system configured to produce the fermentation product;
a distillation system configured to recover ethanol from the fermentation product;
wherein the fermentation product comprises ethanol produced by fermentation of pentose and ethanol produced from the fermentation of hexose;
wherein the biomass comprises lignocellulosic material; wherein the lignocellulosic material consists essentially of corn cobs, corn plant husks, corn plant leaves and corn plant stalks.

45. The system of claim 44 wherein the liquid component comprises at least 5 percent solids and at least 2 percent xylose by weight.

46. The system of claim 44 wherein the solids component comprises at least 20 percent solids and at least 20 percent cellulose by weight.

47. The system of claim 44 wherein the pentose comprises xylose and the hexose comprises glucose.

48. The system of claim 45 wherein the liquid component comprises at least 4 percent xylose by weight.

49. The system of claim 48 wherein the liquid component comprises at least 0.1 percent glucose by weight.

50. The system of claim 49 wherein the dilute acid supplied to the apparatus for pre-treatment comprises sulfuric acid in a concentration of between about 0.85 and 1.0 percent by weight.

51. A method to pre-treat biomass to be used in a biorefinery to produce a fermentation product comprising the steps of:
preparing the biomass into prepared biomass; and
pre-treating the prepared biomass into pre-treated biomass by application of a dilute acid having a concentration of about 0.8 to 1.1 percent by weight at a temperature of about 130 to about 170 degrees Celsius for a period of time in a range of about 8 to 12 minutes;
wherein the fermentation product can be obtained by separating the pre-treated biomass into a liquid component comprising xylose and a solids component from which glucose can be made available and accessing xylose for fermentation into the fermentation product;
wherein the biomass comprises lignocellulosic material comprising (a) corn cobs, corn plant husks, corn plant leaves and corn stalks and (b) glucan at about 35 to 45 percent by weight and xylan at about 20 to 35 percent by weight;
wherein the prepared biomass comprises at least 10 percent solids by weight in a slurry provided to pre-treatment;
wherein the liquid component comprises xylose in a percentage of at least 80 percent of the xylan in the lignocellulosic material;
wherein the solids component comprises glucan at about 45 to 65 percent by weight and lignin at about 20 to 30 percent by weight;
and wherein the solids component comprises at least 75 percent of the glucan from which glucose can be made available in the lignocellulosic material.

52. The method of claim 51 wherein the liquid component comprises xylose at about 3.5 to 5.2 percent by weight.

53. The method of claim 1 wherein the glucan comprises cellulose and the solids component comprises at least 90 percent of the cellulose in the lignocellulosic material.

54. The method of claim 1 further comprising the step of accessing xylose and glucose for fermentation into the fermentation product.
